(19) United States
(12) Patent Application Publication
(10) Pub. No.: US 2015/0253328 A1
(43) Pub. Date: Sep. 10, 2015

(54) NANOMATERIAL-INDUCED CYTOTOXICITY DETECTION COMPOSITION AND KIT, AND NANOMATERIAL-INDUCED CYTOTOXICITY DETECTION METHOD

(71) Applicant: KOREA UNIVERSITY RESEARCH AND BUSINESS FOUNDATION, Seoul (KR)

(72) Inventors: Sang-Wook Son, Seoul (KR); Sang Hoon Jeong, Seoul (KR)

(73) Assignee: KOREA UNIVERSITY RESEARCH AND BUSINESS FOUNDATION, Seoul (KR)

(21) Appl. No.: 14/358,224

(22) PCT Filed: Nov. 14, 2012
(86) PCT No.: PCT/KR2012/009615

§ 371 Date: May 14, 2014

(30) Foreign Application Priority Data
Nov. 14, 2011 (KR) 10-2011-0118266

(51) Int. Cl. G01N 33/573; G01N 33/68

(52) U.S. Cl. CPC G01N 33/573; G01N 33/6875; G01N 2333/912; G01N 2333/4703; G01N 2333/525

(57) ABSTRACT

The present invention concerns a detection marker which is specific to nanomaterial-induced cytotoxicity, and provides: a nanomaterial-induced cytotoxicity detection composition and kit, comprising an ERK gene which is specifically overexpressed when nanomaterial-induced cytotoxicity is present, an Egr-1 gene which is activated by the ERK gene, and an agent for measuring the level of the mRNA thereof or protein thereof; and a method for detecting nanomaterial-induced cytotoxicity by using the composition and kit. The composition, the kit and the detection method according to the present invention allow straightforward and accurate detection of cytotoxicity induced by nanomaterials comprised in, for example, the cells of nanomaterials.

### TECHNICAL FIELD

The present invention relates to a composition, kit and method for detecting nanomaterial-induced cytotoxicity in a biological sample. More specifically, the present invention relates to a method capable of detecting nanomaterial-induced cytotoxicity in a biological sample in a quick and simple manner.

### BACKGROUND ART

In recent years, nanomaterials have received a lot of attention in the social and scientific fields due to their utility. Nanomaterials are actually applied in many products; for example, silver nanomaterials are added to washing machines or detergents. Among them, nanomaterials having fluorescence emission values have been actively studied as new diagnostic technology in the medical world. However, a question about the safety of nanomaterials has been posed, and problems for the authority for use of products containing nanomaterials have been caused, and thereby studies on the toxicity of nanomaterials have been conducted. However, these studies are still insufficient, and particularly, there are no criteria for evaluating the toxicity of nanomaterials, and thus the toxicity of nanomaterials is becoming more problematic.

Accordingly, there is a need for the development of a method capable of measuring the toxicity of nanomaterials in a simpler and more accurate manner within a short time by examining the protein and RNA levels caused by the toxicity of cell-exposed nanomaterials by an experimental method using cells.

Patent publications related to the background of the present invention include Korean Patent Application Nos. 2009-0003356 and 2008-0094939.

### DISCLOSURE OF INVENTION

### Technical Problem

The present invention has been made in order to solve the above-described problems occurring in the prior art, and it is an object of the present invention to provide a composition and method capable of simply and accurately detecting nanomaterial-induced cytotoxicity in cells.

### Technical Solution

To achieve the above object, the present invention provides a composition for detecting nanomaterial-induced cytotoxicity, which comprises an agent for measuring the mRNA or protein level of ERK gene that is specifically overexpressed in the presence of nanomaterials or Egr-1 gene that is activated by the ERK gene.

In an embodiment of the present invention, the agent for measuring the level of the protein may comprise an antibody specific to the protein.

In another embodiment of the present invention, the agent for measuring the level of the protein may be a composition for immunohistochemical staining, which is capable of detecting the expression of the Egr-1 protein in a tissue.

The present invention also provides a kit for detecting nanomaterial-induced cytotoxicity, which comprises the above composition.

In an embodiment of the present invention, the kit may be an RT-PCR kit, a DNA chip kit or a protein chip kit.

The present invention also provides a method for detecting nanomaterial-induced cytotoxicity, the method comprising the steps of: measuring the expression level of ERK gene in a biological sample ex vivo, the expression level of Egr-1 gene that is activated by the ERK gene, or the level of a protein that is encoded by the gene; and comparing the expression level of the gene or the level of the protein, which is encoded by the gene, with the expression level of the corresponding gene in a normal control sample or the level of the corresponding protein.

In an embodiment of the present invention, the expression level of the gene may be determined by measuring the mRNA level of the gene. For example, a method for measuring the mRNA level may be performed by reverse transcriptase polymerase chain reaction, competitive reverse transcriptase polymerase chain reaction, real-time reverse transcriptase polymerase chain reaction, RNase protection assay, Northern blotting, or DNA chip assay.

In another embodiment of the present invention, a method for measuring the level of the protein may be performed using any one of a method using antibody, Western blotting, ELISA, radioimmunoassay, radical immunodiffusion, ouchterlony immunodiffusion, rocket immunoelectrophoresis, immunohistochemical staining, immunoprecipitation assay, complement fixation assay, FACS, and protein chip assay.

### Advantageous Effects

The present invention has an advantage in that nanomaterial-induced cytotoxicity can be detected in a rapid, simple and accurate manner.

### BRIEF DESCRIPTION OF THE DRAWINGS

FIG. 1 shows the results of analyzing the characteristics of ZnO.

FIGS. 2a and 2b show the results of measuring the absorbance of cells treated with ZnO in a dose-dependent manner. The horizontal axis indicates concentration, and the vertical axis indicates cell viability.

FIG. 3 shows the characteristics of oxidative stress formation in cells treated with ZnO-NPs.

FIG. 4 shows the bands of Egr-1, p-ERK, ERK and b-actin proteins in ZnO-treated cells in a time-dependent manner.

FIG. 5 shows the expression levels of Egr-1 in ZnO-treated cells at varying time points.

FIG. 6 shows the expression levels of P-ERK in ZnO-treated cells at varying time points.

FIG. 7 shows the protein bands of Egr-1 in cells, treated with both ZnO(−) and MAPK inhibitor, at varying time points.

FIG. 8 shows the protein bands of Egr-1 and B-actin in cells, treated with both ZnO(+) and MAPK inhibitor, at varying time points.

FIG. 9 shows the expression level of Egr-1 in cells treated with both ZnO(+) and MAPK inhibitor.

FIG. 10 shows the expression level of Egr-1 in cells treated with both ZnO(−) and MAPK inhibitor.

FIG. 11 shows the expression pattern of Egr-1 protein in cells treated with ZnO-NPs.

FIG. 12 shows the expression patterns of P-Erk, Erk, P-JNK, JNK1, P-p38 and p38 in cells treated with ZnO-NPs.

FIG. 13 shows the expression pattern of Egr-1 after treatment with both ZnO-NPs and MAPK inhibitor or ROS inhibitor.

FIGS. 14 and 15 show the inhibition of TNF-α by Egr-1 siRNA in cells treated with ZnO-NPs.

FIG. 16 shows the results of inducing the expression of Egr-1 and TNF-α by treating the mouse skin with ZnO.

### BEST MODE FOR CARRYING OUT THE INVENTION

Hereinafter, the present invention will be described in detail.

The present invention is directed to a marker of ERK gene or Egr-1 gene that is activated by ERK gene for detecting nanomaterial-induced cytotoxicity.

As used herein, the term “detecting” refers to checking whether nanomaterial-induced cytotoxicity is present in a sample, for the purpose of the present invention.

As used herein, the term “nanomaterials” refers to nano-scale materials having a side length of 100 nm or less when viewed three-dimensionally. Specific examples of the nanomaterials include, but are not limited to, ZnO, TiO₂, gold nanoparticles, silver nanoparticles, quantum dots, polystyrene, nano silica, etc.

As used herein, the term “marker for detection”, “marker for detecting” or “detection marker” refers to a material by which cells showing nanomaterial-induced cytotoxicity can be distinguished from cells showing no cytotoxicity. Examples of the marker include organic biomolecules such as polypeptides, nucleic acids (e.g., mRNA etc.), lipids, glycolipids, glycoproteins, saccharides (monosaccharides, disaccharides, oligosaccharides, etc.) and the like, which show increased expression pattern in cells showing nanomaterial-induced cytotoxicity. For the purpose of the present invention, the marker for detection of nanomaterials is ERK gene or Egr-1 gene that is activated by ERK gene, the expression of which increases in the presence of nanomaterials.

Specifically, the present invention is directed to a composition for detecting nanomaterials, which comprise an agent for measuring the mRNA level of ERK gene or Egr-1 gene that is activated by the ERK gene, or the level of the protein thereof.

As used herein, the expression “measuring the mRNA expression level” means checking the presence and expression level of mRNA of nanomaterial marker genes in a biological sample in order to detect nanomaterials. Analysis method for measuring the mRNA expression level include, but are not limited to, reverse transcriptase polymerase chain reaction (RT-PCR), competitive reverse transcriptase polymerase chain reaction (competitive RT-PCR), real-time reverse transcriptase polymerase chain reaction (real-time RT-PCR), RNase protection assay, Northern blotting, and DNA chip assay.

As used herein, the expression “measuring the protein expression level” means checking the presence and expression level of a protein expressed from a nanomaterial marker gene in a biological sample in order to detect nanomaterial-induced cytotoxicity. Preferably, the level of a protein of the marker gene can be checked using an antibody that binds specifically to the protein. Analysis methods for measuring the protein level include Western blotting, etc.

As used herein, the term “antibody” means a specific protein molecule directed to an antigenic site. For the purpose of the present invention, the term “antibody” refers to an antibody that binds specifically to the marker protein, and includes all polyclonal antibodies, monoclonal antibodies and recombinant antibodies.

As described above, because the nanomaterial marker protein is investigated, production of an antibody using the marker protein can be easily performed according to a technique generally known in the art.

A polyclonal antibody may be produced by a method generally known to one skilled in the art, which comprises injecting the nano-material marker protein antigen into an animal, and collecting the blood from the animal to obtain blood serum comprising the antibody. Such polyclonal antibodies may be prepared from any animal hosts including a goat, a rabbit, sheep, a monkey, a horse, a pig, a cow, a dog, etc.

A monoclonal antibody may be produced by a hybridoma method (see Kohler and Milstein (1976) European Journal of Immunology 6:511-519), or phage antibody library (see Clackson et al, Nature, 352:624-628, 1991; Marks et al, J. Mol. Biol., 222:58, 1-597, 1991) generally known in the art. The antibody produced by the above-described method can be isolated and purified by various methods, including gel electrophoresis, dialysis, salt precipitation, ion exchange chromatography, and affinity chromatography, etc.

The antibodies of the present invention include not only a complete form having two full-length light-chains and two full-length heavy chains, but also functional fragments of the antibody molecule. The functional fragments of the antibody molecule as used herein refers to fragments that retain an antigen binding function, such as Fab, F(ab′), F(ab′)2 and Fv, etc.

In addition, the composition for immunohistochemical staining according to the present invention may comprise: an antibody that binds specifically to Egr-1 or its soluble fragment, a secondary antibody conjugate with a label that color-reacts with a substrate; a substrate solution which develops a color reaction with the label; a washing buffer; and a reaction stop buffer, etc. The label conjugated to the secondary antibody is preferably a common coloring agent which can bring about a color change, and includes fluorescein such as HRP (horseradish peroxidase), alkaline phosphatase, colloid gold, fluorescein such as FITC (poly L-lysine-fluorescein isothiocyanate) or RITC (rhodamine-B-isothiocyanate), and dye, etc. The substrate solution is preferably selected depending on the label, and examples thereof include TMB (3,3′,5,5′-tetramethyl bezidine), ABTS [2,2′-azino-bis(3-ethylbenzothiazoline-6-sulfonic acid)], and OPD (o-phenylenediamine), etc. The coloring substrate is preferably provided in the dissolved form in buffer (0.1M NaOAc, pH 5.5). The washing buffer preferably contains phosphate buffer, NaCl and Tween 20. More preferably, the washing buffer is a solution containing 0.02M phosphate buffer, 0.13M NaCl, and 0.05% Tween 20 (PBST). After the antigen-antibody combining reaction, the antigen-antibody complex is treated with the secondary antibody conjugate, and added to immobilized body in suitable amounts and then washing 3-6 times with the washing buffer. A sulfuric acid solution may be used as the reaction stop buffer.

The present invention is also directed to a kit for detecting nanomaterial-induced cytotoxicity, which comprises the above-described composition. The kit may be an RT-PCR kit, a DNA chip kit or a protein chip kit.

Preferably, the kit for detecting nanomaterial-induced cytotoxicity may comprise a composition, solution or device comprising one or more components suitable for an analysis method.

The diagnostic kit may be a general kit that is used to detect toxicity, and can comprise essential elements required to perform reverse transcriptase polymerase chain reaction.

The present invention also provides a method of detecting nanomaterial-induced cytotoxicity using the above-described composition for detecting nanomaterial or the above-described kit for detecting nanomaterial-induced cytotoxicity.

In a specific aspect, the present invention is directed to a method for detecting nanomaterial-induced cytotoxicity in a cell, the method comprising the steps of: measuring the expression level of ERK gene or Egr-1 gene that is activated by the ERK gene, or the level of the protein that is encoded by the gene in a cell ex vivo; and comparing the expression level of the gene or the level of the protein which is encoded by the gene, with the expression level of the corresponding gene or the level of the corresponding protein in a normal control sample.

As used herein, the term “biological sample” is meant to include, but not limited to, samples such as tissue, cell, whole blood, serum, plasma, salvia, sputum, phlegm, cerebrospinal fluid or urine, which show the change in expression level of the nanomaterial marker gene by nanomaterials.

Analysis methods for measuring the mRNA level include, but are not limited to, reverse transcriptase polymerase chain reaction, competitive reverse transcriptase polymerase chain reaction, real-time reverse transcriptase polymerase chain reaction, RNase protection assay, Northern blotting, and DNA chip assay, etc.

Through the above detection methods, the expression of mRNA in the sample to be detected can be compared with the expression of mRNA in the normal control sample, and nanomaterial-induced cytotoxicity can be detected by determining whether the expression of the nanomaterial marker gene into mRNA significantly increased.

Measurement of the mRNA expression level is preferably performed by a reverse transcriptase polymerase chain reaction using primers specific to the nanomaterial marker gene or by a DNA chip assay.

In the case of the reverse transcriptase polymerase chain reaction, the resulting PCR product is electrophoresed, and the pattern and thickness of the band are confirmed, thereby the expression and level of the gene used as the nanomaterial detection marker can be determined. When the expression level is compared with that of the control group, whether the level of nanomaterials in the sample is normal or abnormal can be easily determined.

Whereas, the DNA chip assay employs a DNA chip in which a nucleic acid corresponding the nanomaterial marker gene or its fragment is attached to a substrate like glass at high density. In this DNA chip assay, mRNA is isolated from the sample, and a cDNA probe labeled with a fluorescent substance is hybridized to the DNA chip. Then, whether nanomaterials in the sample are cytotoxic can be determined.

Isolation process of protein from the biological sample can be performed using a known process, and the protein level can be measured by various methods.

Methods for measuring the protein level include, but are not limited to, a method using antibody, Western blotting, radioimmunoassay, radical immunodiffusion, ouchterlony immunodiffusion, rocket immunoelectrophoresis, immunohistochemical staining, immunoprecipitation assay, complement fixation assay, FACS, and protein chip assay, etc.

Preferably, the protein level can be measured by Western blotting using at least one antibody specific to the nanomaterial marker. Specifically, total protein is isolated from the sample and electrophoresed to separate the proteins according to size. Then, the proteins are transferred to a nitrocellulose membrane and reacted with an antibody. The amount of the produced antigen-antibody complex is analyzed using a labeled antibody to determine the amount of protein produced by expression of the gene, thereby detecting nanomaterials. The detection method is conducted by analyzing the expression level of the marker gene in the control group and the expression level of the marker gene in cells containing an excessive amount of nanomaterials. The mRNA or protein level can be expressed as the absolute difference (e.g., Erg-1/β-actin) or relative difference (e.g., relative intensity of signals) of the marker protein.

In another preferred embodiment, immunohistochemical staining is performed using at least one antibody specific to the nanomaterial marker.

A normal tissue and a tissue suspected of containing an excessive amount of nanomaterials are collected and fixed, and then paraffin-embedded blocks are prepared. These blocks are sectioned to a thickness of several um, and the sections are attached to a glass slide and reacted with an antibody selected from among the above-described antibodies according to a known method. Then, unreacted antibody is washed out, and the sections are labeled with one of the above-described detection level, and labeling with the antibody is observed under a microscope. Herein, the expression level of TNF-α can also be analyzed to determine whether nanomaterials cause inflammatory reactions.

In still another embodiment, a protein chip is used, in which one or more antibodies specific to the nanomaterial marker are arranged at a predetermined position on a substrate and immobilized at high density. In a method of analyzing a sample using the protein chip, protein is isolated from the sample, and the isolated protein is hybridized to the protein chip to form an antigen-antibody complex. Then, the presence or expression level of the protein in the sample is analyzed to determine whether the sample contains an excessive amount of nanomaterials.

### EXAMPLES

Hereinafter, the present invention will be described in further detail with reference to examples. It will be obvious to a person having ordinary skill in the art that these examples are illustrative purposes only and are not to be construed to limit the scope of the present invention.

### Example 1

### Measurement of Influence of ZnO Nanoparticles

### Experimental Example 1

### Analyzing ZnO Characterization by Electron Microscope

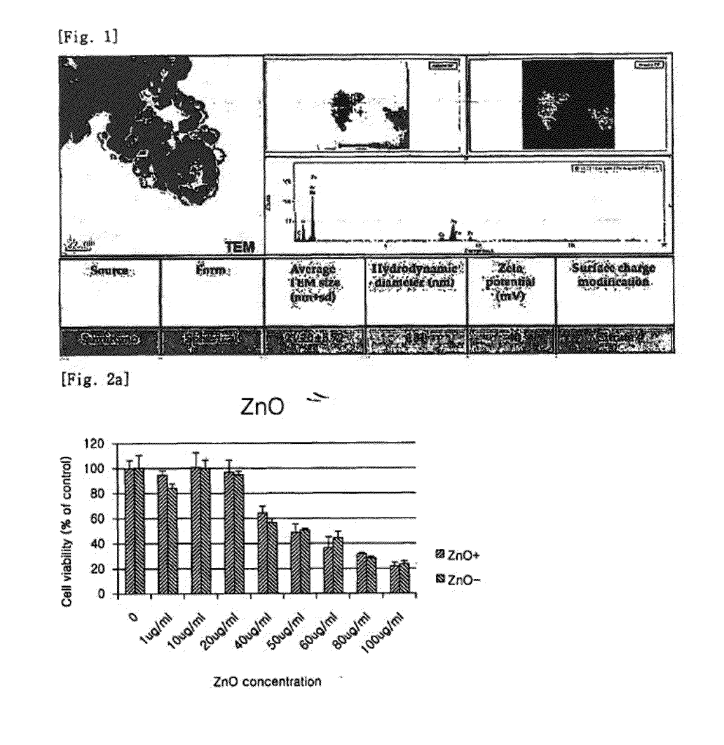

In order to examine the characteristics of the nanomaterial ZnO by a transmission electron microscope (TEM), ZnO was diluted in PBS to a concentration of about 10 ng/ml, and about 2 μl of the dilution was dropped onto a 200-mesh copper grid with carbon coating, and then dried in an oven at 60° C. for 15 minutes, followed by observation with a transmission electron microscope. In order to examine whether ZnO used in this Example is correct ZnO, the ZnO was analyzed by energy dispersion x-ray spectroscopy. The characteristics of ZnO are shown in FIG. 1.

### Experimental Example 2

### Cell Viability Assay

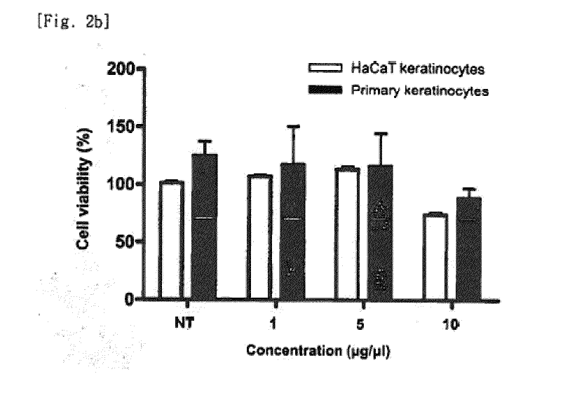

Human keratinocyte (HaCaT) cells and human epidermal keratinocytes-neonatal (HEKn; primary keratinocytes) were prepared at a concentration of 1×10³ cells/100 μl in 96-well culture plates. In the case of HaCaT, 100 ul of DMEM medium containing 10% FBS and 1% penicillin/streptomycin was added, and in the case of HEKn, 100 ul of a medium containing growth factors was added, and the cells were cultured in a CO₂ incubator for 24 hours, and then treated with ZnO in a dose-dependent manner. Then, 10 ul of a cell viability measurement solution CCK-8 (Dojindo Company) was added to each well, respectively, followed by incubation in a CO₂ incubator for 4 hours. In view of the fact that ZnO interferes with the measurement of absorbance, 50 ul of the cell culture was transferred to each well of fresh 96-well plates, and then the absorbance at a wavelength of 450 nm was measured using a microplate reader. The results of the measurement are shown in FIGS. 2a and 2b.

As shown in FIGS. 2a and 2b, ZnO induces cytotoxicity to the cell.

### Experimental Example 3

### Measurement of Reactive Oxygen species (ROS) that were changed by nanoparticles (ZnO-NPs)

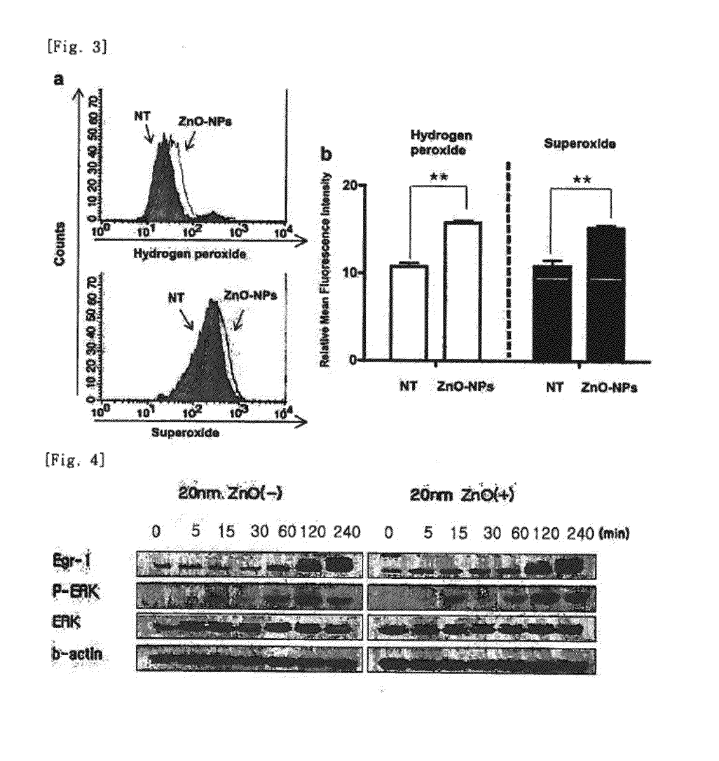

Human keratinocytes (HaCaT) were cultured in a 6-well culture plate in a CO₂ incubator for 24 hours using a DMEM medium containing 10% FBS and 1% penicillin/streptomycin, followed by treatment with 10 ul of ZnO. Then, in order to measure cytosolic and mitochondrial reactive oxygen species produced in the cells by ZnO, the cells were treated with each of 10 nM of 2,7-dichlorofluorescein diacetate (DCFDA, Invitrogen) and 5 uM of MitoSOX™ (Invitrogen) and incubated in a CO₂ incubator for 20 minutes. Then, the cells washed with HBSS, stained and analyzed by fluorescence activated cell sorter (FACS). The results of the analysis are shown in FIG. 3. FIG. 3a shows the results of FACS analysis performed to measure ROS in the HaCaT cells treated with 10 μg/ml of ZnO-NPs for 30 minutes. FIG. 3b is a bar graph showing the mean fluorescence intensity of ROS (hydrogen peroxide) by DCF-DA staining and mitochondrial ROS (superoxide) by MitoSOX™ staining (** (P<0.01) indicates a statistically significant difference compared to control (no treatment)).

### Example 2

### Western Blot Assay

HaCaT cells were dispensed in a 60-mm cell culture dish and treated with a DMEM medium containing 10% FBS and 1% penicillin/streptomycin (P/S), after the cells were cultured in a CO₂ incubator for 24 hours. Then, 2 ml of DMEM medium (Hyclone Company) free of FBS and P/S was added to the cells, followed by incubation in a CO₂ incubator for 18 hours. Then, the cells were treated with 10 ug/ml of ZnO in a time-dependent manner (0, 5, 15, 60, 120 and 240 minutes), and then the medium was removed from the plate. The cells in the plate were treated with 1 ml of 1×PBS, scrubbed with a scrapper, transferred to a 1.5-ml tube, and then centrifuged. The supernatant was discarded and the cell pellets were collected. The cell pellets collected in a time-dependent manner were treated with RIPA cell lysis buffer, vortexted three times on ice for 10 minutes each time, and then centrifuged at 4° C. and 14,000 rpm for 30 minutes. The supernatants were transferred to other 1.5-ml tubes and quantified by BCA assay (Gendepot Company).

The quantified samples were transferred to other 1.5-ml tubes to make the same amount of samples, and 5× loading buffer was added to the samples so that the total amounts were equal (total 1×). Then, each of the samples was incubated at 95° C. for 5 minutes. Next, 27 ug of each sample was loaded onto bisacrylamide gel for protein separation and electrophoresed (Bio-rad Company).

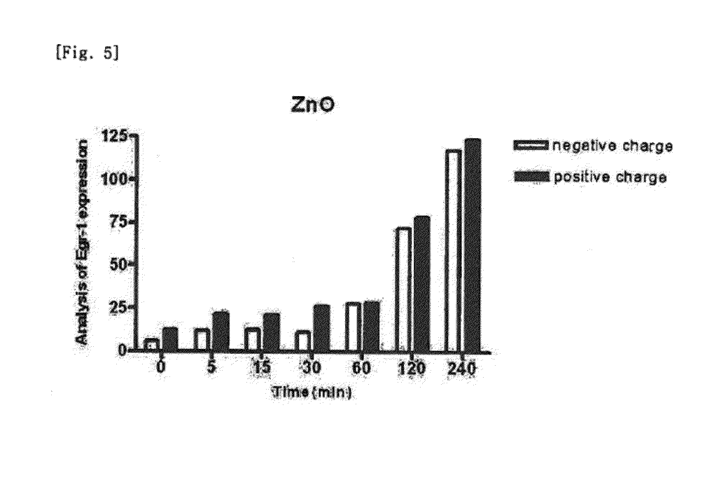

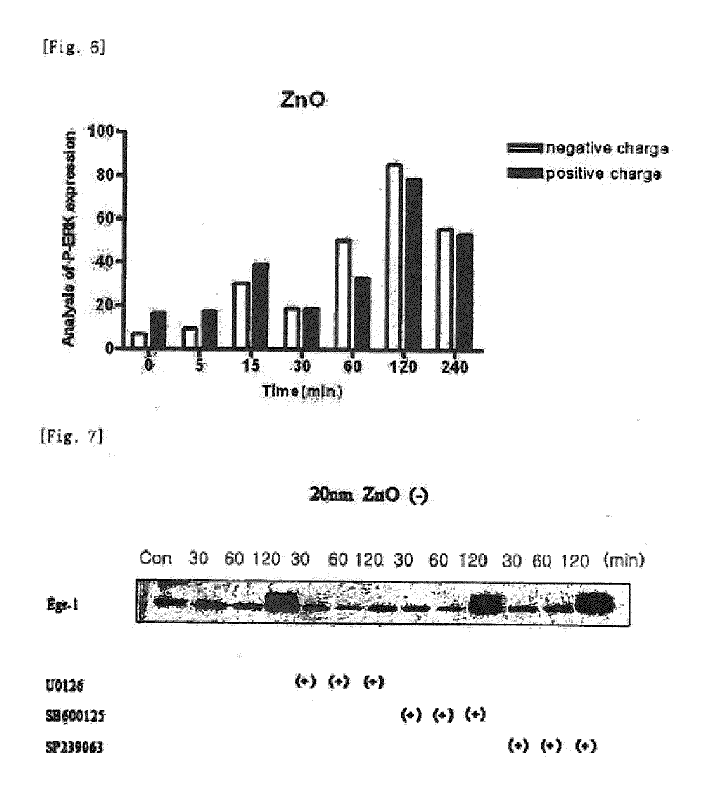

After completion of the electrophoresis, each sample was transferred to a PVDF membrane (Bio-rad Company) and blocked in a mixture (1:1 w/w) of skim-milk (non-fat 5%) and TBST for 20 minutes. After completion of blocking, each sample was incubated overnight at 4° C. with a 2000:1 dilution of each of Egr-1, β-actin primary antibody (Sigma) and p-ERK, ERK primary antibody (Cell Signaling) in a mixture (1:1 w/w) of skim-milk (non-fat 5%) and TBST. Next day, each sample was washed three time with 1×TBST for 5 minutes each time and incubated with a 2000:1 rabbit secondary antibody of each of Egr-1, p-ERK and ERK and a 2000:1 mouse secondary antibody of β-actin in a mixture (1:1 w/w) of skim-milk (non-fat 5%) and TBST for 1 hour. Then, each sample was washed three times for 10 minutes each time, and each protein band was obtained using a film. The bands of Egr-1, p-ERK, ERK and b-actin at varying time points are shown in FIG. 2. In addition, FIGS. 5 and 6 show the expression levels of Egr-1 and P-ERK at varying time points, respectively.

As can be seen in FIGS. 4 to 6, when the cells were treated with 20 nm ZnO, the expression levels of Egr-1 protein and ERK protein increased based on time. This suggests that Egr-1 or ERK is useful as a marker protein for detecting nanomaterial-induced cytotoxicity.

### Example 3

### MAPK Inhibitor Assay

A Western blot assay was performed in the same manner as described in Example 2, except for the following differences. At 30 minutes before treatment with ZnO, cells were treated with an MAPK (mitogen-activated proteinkinase) inhibitor (U0126: ERK inhibitor, SB600125: p38 inhibitor, SP239063: JNK inhibitor-sigma product), and after 30 minutes, the cells were treated with 2 ml of fresh DMEM medium free of FBS and P/S. After 2 hours from treatment with ZnO, the medium was removed, and the cells were treated with 1×PBS, scraped with a scrapper, and then transferred to a 1.5 ml tube.

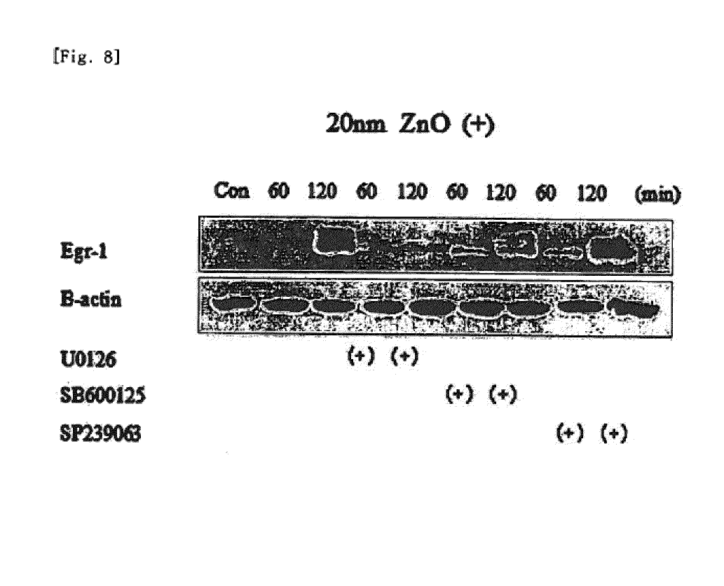

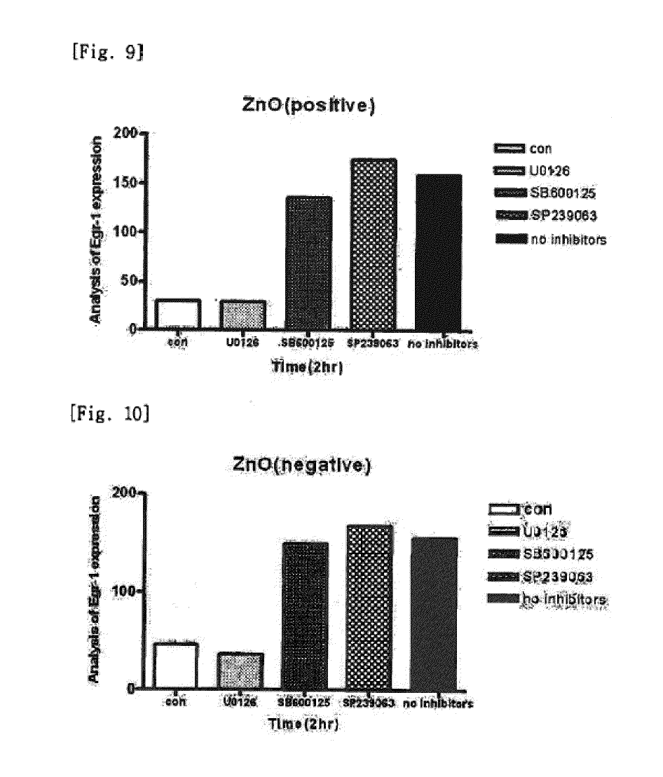

The bands of Egr-1 and b-actin proteins by treatment with both ZnO and the MAPK inhibitor at varying time points are shown in FIGS. 7 and 8. In addition, the expression levels of Egr-1 by treatment with both ZnO and the MAPK inhibitor are shown in FIGS. 9 and 10.

As shown in FIGS. 7 to 10, the expression of the Egr-1 protein in the group treated with the ERK inhibitor U0126 significantly decreased. This suggests that the increase in the expression of the marker protein Egr-1 is attributable to the ERK signal.

### Example 4

### Analysis of Erg-1 Protein Expression Pattern Induced by ZnO-NPs

### Experimental Example 1

### Luciferase Activity Assay

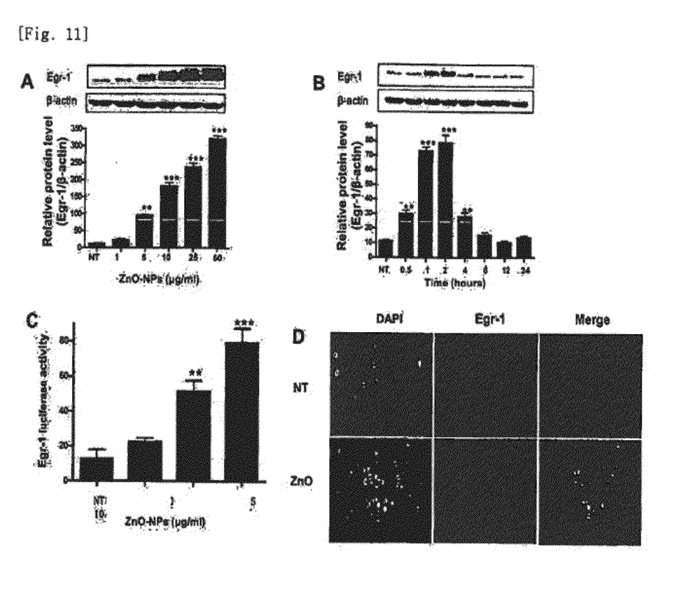

An Egr-1 promoter having a length of 1000 base pairs (bp) was synthesized from genomic DNA by a polymerase chain reaction (PCR) and was ligated into a pGL4.17 luciferase reporter vector containing a luciferase gene using a restriction enzyme. The vector was transformed into E. coli to obtain a large amount of the promoter. Human keratinocyte (HaCaT) cells were dispensed into a 60-mm cell culture dish and treated with DMEM medium containing 10% FBS and 1% penicillin/streptomycin (P/S), followed by incubation in a CO₂ incubator for 24 hours. Then, 2 ml of DMEM medium (Hyclone Company) free of FBS and P/S was added to the cells. Meanwhile, 1 ug of the synthesized vector was added to 100 ul of OPTI-MEM medium, and 3 ul of lipofectamine 2000 (Invitrogen Company) as a transfection reagent was added to 97 ul of OPTI-MEM medium, and after 5 minutes, the two mixtures were combined with each other and allowed to stand for 20 minutes. Next, the synthesized vector mixture was dispensed in the above-prepared 60-mm cell culture dish and well shaken. After 5 hours, the existing medium was removed, the cells were treated with 2 ml of 10% FBS-containing DMEM, and 48 hours, treated with varying concentrations of ZnO. After 24 hours, the medium was removed, and the cells in the plate were treated with 1 ml of 1×PBS, scraped with a scrapper and transferred to 1.5-ml tubes, followed by centrifugation. The supernatant was removed, and cell pellets were collected. The cell pellets were treated with cell lysis buffer for luciferase and vortexed three times on ice for 10 minutes each time, followed by centrifugation at 4° C. and 14,000 rpm for 30 minutes. Next, 50 ul of the sample was treated with luciferin capable of emitting light by 200 ul of luciferase, and the absorbance of the sample was measured using a luciferase reader (Glomax2000, Promega). As shown in the results in FIG. 11c, the transcription of Egr-1 was regulated in a concentration-dependent manner. As shown in FIG. 11c, the promoter of pEGR(−998/+170)/Luc was activated by ZnO-NPs. The cells were transformed with pEGR(−998/+170)/Luc, and then treated with 1, 5 and 10 μg/ml of ZnO-NPs for 24 hours, and the luciferase activity was measured with a luminometer.

### Experimental Example

### Immunoflourescense Assay

HaCaT cells were dispensed in a 6-well culture plate equipped with a cover glass, and were then treated with a DMEM medium containing 10% FBS and 1% penicillin/streptomycin (P/S), followed by culture in a CO₂ incubator for 24 hours. Next, 2 ml of DMEM medium (Hyclone Company) free of FBS and P/S was added to the cells, followed by incubation in a CO₂ incubator for 18 hours. Then, the medium was completely removed, and the cells were washed three times with 1×PBS and treated with 1 ml of 3.8% formaldehyde for 10 minutes. After removing the formaldehyde, the cells were washed three times in the same manner and treated with 1 ml of 0.1% triton-X100, followed by washing. Then, 2% BSA was added to the cells, followed by blocking at 37° C. for 1 hour. After washing, the cells were incubated with a 1:500 dilution of Egr-1 primary antibody in 1% BSA-containing 0.1% triton-X100 at 37° C. for 1 hour. After washing, the cells were treated with a 1:500 dilution of secondary antibody at 37° C. for 30 minutes. After washing, the cover glass was dried completely and fixed onto a slide glass using a mount solution, followed by observation with a fluorescence microscope. As shown in FIG. 11d, Egr-1 was much expressed in the cellular nuclei, suggesting that the transcription of Egr-1 was regulated. As shown in the results in FIG. 11d, the expression of Egr-1 was detected after stimulating the HaCaT cells with ZnO-NPs for 2 hours. Herein, the cells were incubated with Egr-1 antibody, and the signal was detected by Cy3 anti-rabbit (red) immunofluorescence. DAPI (blue) was added together with secondary antibody to label the nuclei (**P<0.01, ***P<0.001 compared with untreated samples).

### Experimental Example 3

### Western Blot Assay

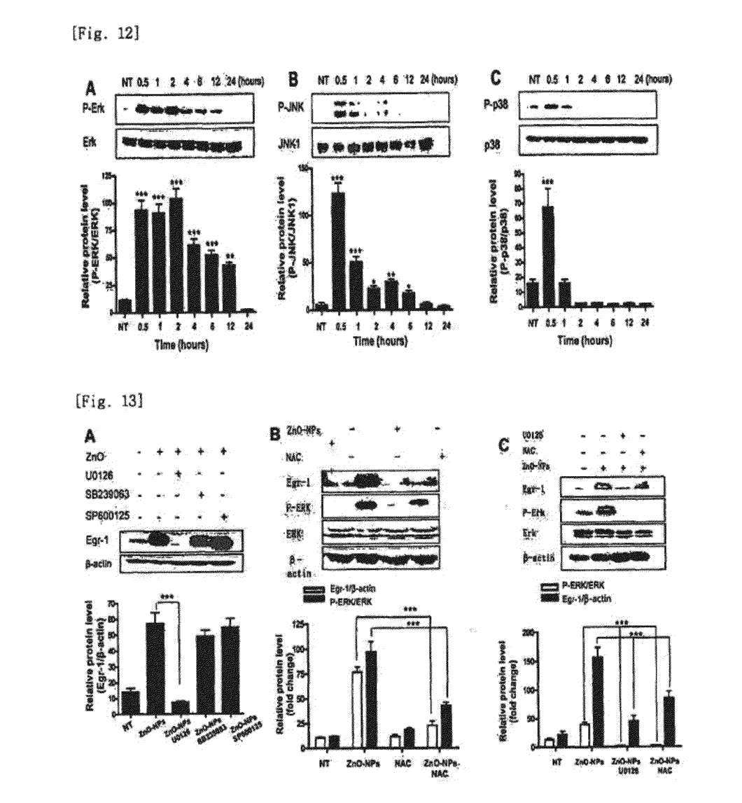

This Example was performed in the same manner as described in Example 2. Namely, cells were treated with 1-50 μg/ml of ZnO-NPs for 2 hours and stimulated with ZnO-NPs, and then the expression of Egr-1 was measured by Western blot assay using anti-Egr-1 antibody. The results of the measurement are shown in FIGS. 11a and 11b. Thus, the expressions of bands of Egr-1 protein at varying concentrations and varying time points can be seen in FIGS. 11a and 11b, respectively. In addition, protein bands for p-ERK, ERK, p-JNK, JNK, p-p38, p38 and b-actin at varying time points are shown in FIGS. 12a, 12b and 12c, respectively. The expression of MAP kinase in HaCaT cells as shown in FIGS. 12a, 12b and 12c was analyzed by Western blot assay using total protein extract and antibody.

### Experimental Example 4

### MAPK Inhibitor Assay

A Western blot assay was performed in the same manner as described in Example 2, except for the following differences. At 30 minutes before treatment with ZnO, cells were treated with an MAPK (mitogen-activated proteinkinase) inhibitor (U0126: ERK inhibitor, SB600125: p38 inhibitor, SP239063: JNK inhibitor-sigma product), and after 30 minutes, the cells were treated with 2 ml of fresh DMEM medium free of FBS and P/S. After 2 hours from the treatment with ZnO, the medium was removed, and the cells were treated with 1×PBS, scraped with a scrapper, and then transferred to a 1.5 ml tube.

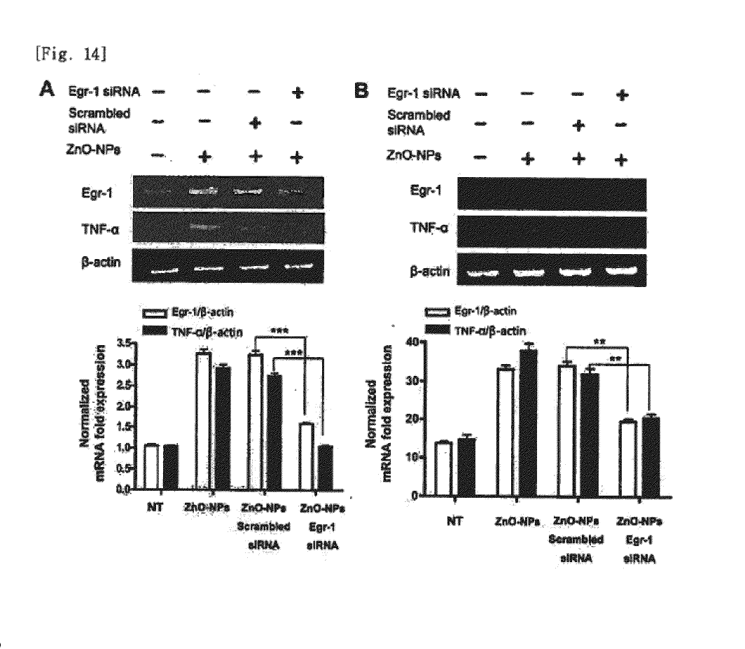

FIG. 13A shows the protein bands of Egr-1 and b-actin by treatment with both ZnO and the MAPK inhibitor at varying time points. As shown in FIG. 13A, the expression of the Egr-1 protein in the group treated with the ERK inhibitor U0126 significantly decreased. However, JNK and a p38 inhibitor did not influence the expression of the Egr-1 protein (*P<0.05; **P<0.01, ***P<0.001 compared with no treated samples). Thus, it can be seen that the increase in the expression of the marker protein Egr-1 is attributable to the ERK signal.

### Experimental Example 5

### ROS Inhibitor Assay

A Western blot assay was performed in the same manner as described in Example 2, except for the following differences. At 2 hours before treatment with ZnO, cultured cells were treated with 10 uM/ml of the antioxidant N-acetyl cystein (NAC), and after 2 hours, the cells were treated with 2 ml of fresh DMEM medium free of FBS and P/S. After 1 hour from the treatment with ZnO, the medium was removed, and the cells were treated with 1×PBS, scraped with a scrapper, and then transferred to a 1.5 ml tube. As shown in FIGS. 13B and 13C, when the cells were treated with the antioxidant NAC, the expression bands of Egr-1 and p-ERK proteins significantly decreased. Thus, it can be seen that the expressions of the marker proteins Egr-1 and p-ERK are increased by ROS (B: HaCaT, C: HEKn). Herein, the data points indicate the β-actin-normalized mean and ERK-normalized mean of three independent experiments compared using two-way Anova (***P<0.001).

### Experimental Example

### Small Interfering RNA (siRNA) Assay

Human keratinocyte (HaCaT) cells and human epidermal keratinocytes-neonatal (HEKn, primary keratinocytes) were dispensed in 60-mm cell culture dishes and treated with a DMEM medium containing 10% FBS and 1% penicillin/streptomycin (P/S), followed by culture in a CO₂ incubator for 24 hours. Then, 2 ml of DMEM medium (Hyclone Company) free of FBS and P/S was added to the cells. Meanwhile, each of Egr-1 siRNA and control scrambled siRNA was added to 100 ul of OPTI-MEM medium to a concentration of 10 nM, and 3 ul of lipofectamine 2000 (Invitrogen Company) as a transfection reagent was added to 97 ul of OPTI-MEM medium, and after 5 minutes, the two mixtures were combined and allowed to stand for 20 minutes. The siRNA mixture was dispensed in the above-prepared 60 mm cell culture dish and well shaken. After 5 hours, the existing medium was removed, and the cells were treated with 2 ml of 10% FBS-containing DMEM medium. After 48 hours, the cells were treated with ZnO for 1 hour for Egr-1 and for 12 hours for TNF-α, followed by removal of the medium. Then, the cells were treated with 500 ul of TRiZol (Invitrogen), scraped with a scrapper, and then transferred to 1.5-ml tubes. Next, each of the tubes was treated with 100 ul of chloroform and strongly shaken, followed by centrifugation at 12,000 rpm for 15 minutes. The supernatant was transferred to other tubes, and it was treated with 250 ul of isopropanol, and then allowed to stand at room temperature for 10 minutes. Next, the content in the tube was centrifuged at 12,000 rpm for 10 minutes, and the supernatant was completely removed, and the cell pellets were treated with 500 ul of a solution of 70% ethanol in DEPC-water and centrifuged for 5 minutes in the same manner. Then, the precipitate (RNA) was dried until it was transparent, and 30 ul of DEPC-water was added thereto. Next, 1 ug of the RNA was synthesized into cDNA using reverse transcriptase, and the cDNA was subjected to a polymerase chain reaction (PCR) using Taq polymerase. The resulting PCR product was electrophoresed on 2% agarose gel, and the bands were examined.

As shown in FIGS. 14A and B, the expression of TNF-α in the sample treated with Egr-1 siRNA significantly decreased, suggesting that the expression of TNF-α is regulated by Egr-1 (A: HaCaT, B: HEKn). In FIG. 14, Egr-1 siRNA or scrambled siRNA was transfected into HaCaT cells using Lipofectamine™ 2000, and the TNF-α mRNA expression level was measured by RT-PCR (a) and ELIZA (b) after stimulating the cells with 10 μg/ml ZnO-NPs for 12 hours (***P<0.001 indicates a statistically significant difference compared to no treated sample (a, b)).

### Experimental Example 7

### Enzyme-Linked Immunosorbent Assay (ELISA)

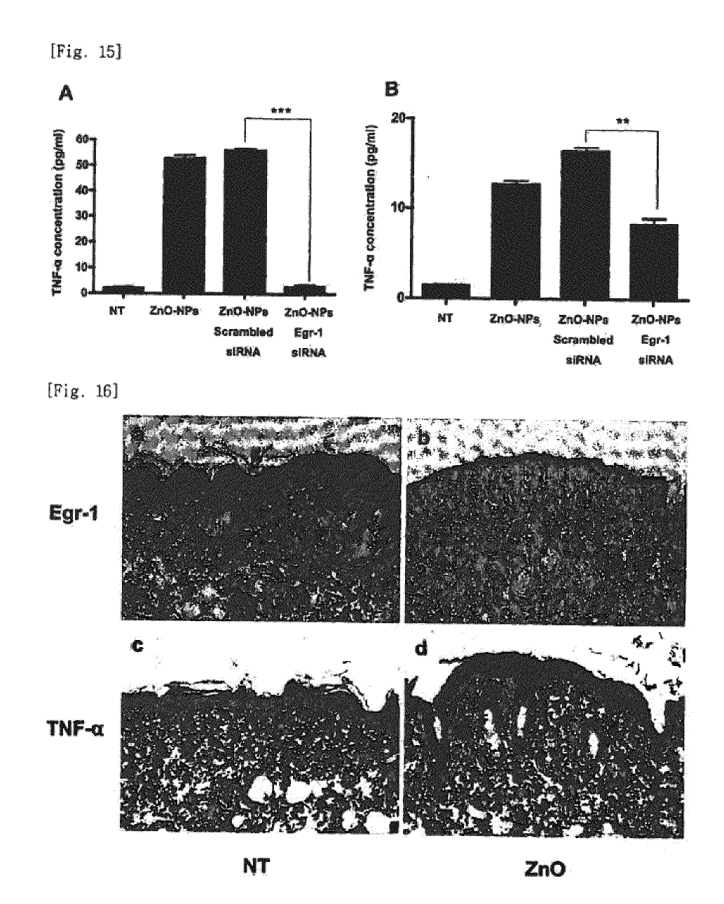

Human keratinocyte (HaCaT) cells and human epidermal keratinocytes-neonatal (HEKn, primary keratinocytes) were dispensed in 60-mm cell culture dishes and treated with a DMEM medium containing 10% FBS and 1% penicillin/streptomycin (P/S), followed by culture in a CO₂ incubator for 24 hours. Then, 2 ml of DMEM medium (Hyclone Company) free of FBS and P/S was added to the cells. Meanwhile, each of Egr-1 siRNA and control scrambled siRNA was added to 100 ul of OPTI-MEM medium to a concentration of 10 nM, and 3 ul of lipofectamine 2000 (Invitrogen Company) as a transfection reagent was added to 97 ul of OPTI-MEM medium, and after 5 minutes, the two mixtures were combined and allowed to stand for 20 minutes. Then, the siRNA mixture was dispensed in the above-prepared 60-mm cell culture dish and well shaken. After 5 hours, the existing medium was removed, and the cells were treated with 2 ml of 10% FBS-containing DMEM medium, and after 48 hours, the cells were treated with ZnO for 12 hours. Then, the medium was collected in 15-ml tubes and stored −70° C. until use. Meanwhile, a 96-well plate was coated with coating buffer for 24 hours and treated with TNF-α antibody, after which it was allowed to stand for 2 hours. After washing, the plate was treated with 50 ul of the concentrated sample and allowed to stand for 2 hours, followed by washing. Then, a substrate was added to the plate, and then a change in the color of the plate was examined, and the absorbance of the plate was analyzed by a micro reader. FIGS. 15A and 15B graphically show the absorbance. As shown therein, the expression of TNF-α in the sample treated with Egr-1 siRNA significantly decreased, suggesting that the expression of TNF-α that was extracellularly secreted was regulated by Egr-1 and caused an inflammatory reaction (***P<0.001 indicates a statistically significant difference compared to no treated sample ZnO-NPs+Scrambled siRNA. (** indicates a statistically significant difference compared to ZnO-NPs+Scrambled siRNA (2<0.01)).

### Experimental Example 8

### Immunohistochemistry Assay

50 mg/ml ZnO was added to a solution of 80% glycerol in 1% citrate/HEPES buffer (pH 7.3), and the backs of purchased nude mice were treated everyday for 3 weeks. Then, the skin was collected from the mice. Immunohistochemical staining of the collected skin was performed by the Korea CFC Pathology Laboratory, and the expressions of Egr-1 and TNF-α in the skin were observed with a microscope. As shown in FIG. 16, the expressions of Egr-1 and TNF-α in the skin of the mouse group treated with ZnO significantly increased compared to those in the untreated group, suggesting that an inflammatory reaction occurred not only in the cell experiment, but also in the mouse experiment that is actually applied.

1. A composition for detecting nanomaterial-induced cytotoxicity, which comprises an agent for measuring the mRNA level of ERK gene or Egr-1 gene that is activated by the ERK gene, or the level of the protein thereof.

2. The composition of claim 1, wherein the agent for measuring the level of the protein comprises an antibody which is specific to the protein.

3. The composition of claim 1, wherein the agent for measuring the level of the protein is a composition for immunohistochemical staining, which is capable of detecting the expression of the Egr-1 protein in tissue.

4. A kit for detecting nanomaterial-induced cytotoxicity, which comprises the composition of claim 1.

5. The kit of claim 4, wherein the kit is an RT-PCR kit, a DNA chip kit or a protein chip kit.

6. A method for detecting nanomaterial-induced cytotoxicity in a cell, the method comprising the steps of:
measuring the expression level of ERK gene or Egr-1 gene that is activated by the ERK gene, or the level of the protein that is encoded by the gene in a cell ex vivo; and
comparing the expression level of the gene or the level of the protein which is encoded by the gene, with the expression level of the corresponding gene or the level of the corresponding protein in a normal control sample.

7. The method of claim 6, wherein the expression level of the gene is determined by measuring the mRNA level of the gene.

8. The method of claim 7, wherein measuring the mRNA level is performed by reverse transcriptase polymerase chain reaction, competitive reverse transcriptase polymerase chain reaction, real-time reverse transcriptase polymerase chain reaction, RNase protection assay, Northern blotting, or DNA chip assay.

9. The method of claim 6, wherein measuring the level of the protein is performed using any one of an antibody-based method, Western blotting, ELISA, radioimmunoassay, radical immunodiffusion, ouchterlony immunodiffusion, rocket immunoelectrophoresis, immunohistochemical staining, immunoprecipitation assay, complement fixation assay, FACS, and protein chip assay.

10. The method of claim 9, wherein measuring the level of the protein is performed by immunohistochemical staining, and the expression of TNF-α is also analyzed.

11. A kit for detecting nanomaterial-induced cytotoxicity, which comprises the composition of claim 2.

12. The kit of claim 12, wherein the kit is an RT-PCR kit, a DNA chip kit or a protein chip kit.

13. A kit for detecting nanomaterial-induced cytotoxicity, which comprises the composition of claim 3.

14. The kit of claim 13, wherein the kit is an RT-PCR kit, a DNA chip kit or a protein chip kit.
